(19) United States
(12) Patent Application Publication
(10) Pub. No.: US 2022/0403162 A1
(43) Pub. Date: Dec. 22, 2022

(54) POLYURETHANE COMPOSITIONS SALTED WITH BISBIGUANIDE

(71) Applicant: Lubrizol Advanced Materials, Inc., Cleveland, OH (US)

(72) Inventors: Alexander V. Lubnin, Copley, OH (US); Devin Webber, Cleveland, OH (US); Naser Pourahmady, San Diego, CA (US); Alec Krienen, Cleveland, OH (US)

(21) Appl. No.: 17/777,180

(22) PCT Filed: Nov. 19, 2020
(86) PCT No.: PCT/US2020/061272

§ 371 Date: May 16, 2022

Related U.S. Application Data
(60) Provisional application No. 62/937,381, filed on Nov. 19, 2019.

(51) Int. Cl. C08L 75/04; C08G 18/12; C08G 18/75

(52) U.S. Cl. CPC C08L 75/04; C08G 18/12; C08G 18/755; C08G 18/758

(57) ABSTRACT

The present subject matter relates to a polyurethane composition comprising a polyurethane with at least one free acid group salted with a biguanide free base.

### TECHNICAL FIELD

The present subject matter relates to polyurethane compositions having at least one acid group salted with a biguanide (e.g., bisbiguanide) free base compound.

### BACKGROUND

There has been an ever-increasing focus on compositions that impart antimicrobial properties to products. Preventing microbial build-up and growth has blossomed into a billion-dollar industry. Some pathogenic bacteria have evolved to become resistant to most, if not all, of the currently available antibiotics on the market. These drug-resistant bacteria present a challenge to the healthcare industry: in 2019, one in 20 patients contracts a healthcare-associated infection due to direct exposure to pathogenic bacteria in the hospital environment. The U.S. Centers for Disease Control and Prevention (CDC) estimates the annual economic impact of these healthcare-associated infections to be $28-34 billion. Furthermore, in the food industry, bacteria infestation related recalls have become more prevalent due to current cleaning methods being insufficient. Even consumers are seeking solutions to this issue with products that impart antibacterial properties to architectural paints and home-care and laundry products.

Current best practices to combat microbial contamination utilize more stringent cleaning regimens and developing novel antibiotics. While enhanced cleaning methods may temporarily decrease the bacterial load of the environment, they do not provide long-term antimicrobial efficacy. Once the cleaning has concluded, the surface is then susceptible to bacterial proliferation. Producing a novel antibiotic may seem like an obvious choice, but bacteria have been shown to evolve resistance mechanisms to antibiotics at an alarmingly fast rate, and these discoveries, too, can become obsolete in time.

Chlorhexidine (1,6-bis(4-chloro-phenylbiguanido)hexane; CAS Number 55-56-1) is a bisbiguanide compound and has the following chemical structure:

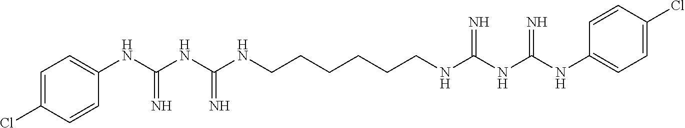 [SMILES: N=C(NCCCCCCNC(=N)NC(=N)Nc1ccc(Cl)cc1)NC(=N)Nc1ccc(Cl)cc1]

Chlorhexidine salts are effective antimicrobial compounds and are commonly used as surgical instrument disinfectants and in hand washes and oral rinses in hospitals and doctors' offices. They are also used to combat biologically active species on medical equipment. In some countries, they are used in topical antiseptics.

Chlorhexidine is found in the market as an approved active pharmaceutical ingredient (API) only in its salt form, such as chlorhexidine digluconate (chlorhexidine gluconate, CHG). Chlorhexidine also exists in a free base form; however, because of its very low solubility in water (0.8 g/L at 20° C., [The Merck Index. 12th Edition. (1996) page 2136]) and susceptibility to hydrolysis (“New stability-indicating high performance liquid chromatography assay and proposed hydrolytic pathways of chlorhexidine.” Yvette Ha and Andrew P. Cheung. Journal of Pharmaceutical and Biomedical Analysis, 14(8), pages 1327-1334 (1996); “Guanidine and Derivatives” Thomas Guthner, Bernd Mertschenk and Bernd Schulz in: Ullmann's Encyclopedia of Industrial Chemistry, vol. 17, pages 175-189 (2012)), the free base is not used for commercial applications where compatibility with water is required.

According to US 2004/0052831 A1 (para. [0008]): “Chlorhexidine is a broad spectrum antimicrobial agent and has been used as an antiseptic for several decades with minimal risk of developing resistant microbes. When relatively soluble chlorhexidine salts, such as chlorhexidine acetate, were used to impregnate catheters, the release was undesirably rapid. The duration of the antimicrobial efficacy of medical devices impregnated with chlorhexidine salts, such as chlorhexidine acetate, is short lived. Chlorhexidine free base is not soluble in water or alcohol and cannot be impregnated in sufficient amounts because of low solubility in a solvent system.”

U.S. Pat. No. 6,897,281 B2 describes breathable polyurethanes, blends, and articles made from polyurethanes having poly(alkylene oxide) side-chain units in an amount from about 12 wt. % to about 80 wt. % of the polyurethane and with less than 25 wt. % of main-chain units of poly(ethylene oxide). The polyurethane of that disclosure includes free carboxylic acid groups which are used as crosslinking sites.

### SUMMARY

The subject matter disclosed herein describes a method of creating an antimicrobial composition by functionalizing polyurethanes having at least one acid group, such as carboxylic acid groups, with a biguanide (e.g., bisbiguanide) free base compound, such as chlorhexidine free base and/or alexidine free base. In some instances herein, chlorhexidine and/or alexidine are described as representatives of biguanides generally (and bisbiguanides in particular), and, as such, it is contemplated that many biguanides will provide the same or similar functionality, properties, etc., as those disclosed herein with regard to chlorhexidine/alexidine, unless explicitly stated otherwise or required by context.

Compositions described herein provide a polymeric salt formed between chlorhexidine free base and polyurethanes, such as nonionically stabilized polyurethane dispersions/solutions and/or anionic polyurethane dispersions/solutions. Chlorhexidine free base's hydrolytic instability and low water solubility make it an unlikely candidate for incorporation into a waterborne system, yet a surprisingly stable and antimicrobially active salt with polyurethanes was formed nonetheless. Without wishing to be bound by theory, it is postulated that the migration of the chlorhexidine free base from its solid phase through the aqueous phase into the polyurethane particle and ensuing salt formation is faster than chlorhexidine's hydrolysis. Thus, a considerable amount of chlorhexidine free base, if not all, survives the journey through the aqueous phase without been hydrolyzed. The polymeric salts of chlorhexidine free base were found to be surprisingly persistent, non-leaching, and durable. It was also found that, when this composition is applied to, such as coated onto, substrates, the chlorhexidine retains its antimicrobial efficacy, killing bacteria on contact and preventing the growth of bacteria on the surface. The latter is even more surprising in the view of chlorhexidine digluconate deactivation by cross-linked poly(acrylic acid) thickener which carries carboxylic groups that are similar to the carboxylic groups in the polyurethanes of the present subject matter (“Inactivation of chlorhexidine gluconate on skin by incompatible alcohol hand sanitizing gels.” N. Kaiser, D. Klein, P. Karanja, Z. Greten, and J. Newman. American Journal of Infection Control, vol. 37, No. 7, pp. 569-573 (2009)).

Chlorhexidine belongs to a class of biguanides, namely bisbiguanides. The mechanism of the biguanide moiety's action relies on dissociation and release of the positively charged biguanide cation. Its bactericidal effect is a result of the binding of this cationic species to negatively charged bacterial cell walls. At low concentrations of chlorhexidine, this results in a bacteriostatic effect; at high concentrations, membrane disruption results in cell death. (“Chlorhexidine Gluconate” on page 183 in: Poisoning and Toxicology Handbook (4th Edn.), Jerrold B. Leikin and Frank P. Paloucek, Eds. (2008), Informa Healthcare USA, Inc.)

In the view of this mechanism, in certain embodiments, it is anticipated that other biguanide and bisbiguanides can replace chlorhexidine in part or in whole. These are disclosed in “Structural Requirements of Guanide, Biguanide, and Bisbiguanide Agents for Antiplaque Activity.” J. M. Tanzer, A. M. Slee, and B. A. Kamay. Antimicrobial Agents and Chemotherapy, 12(6), pp. 721-729 (1977), and U.S. Pat. No. 4,670,592. Examples include, but are not limited to: alexidine, polyhexanide (polyhexamethylene biguanide, PHMB), polyaminopropyl biguanide (PAPB), and the like.

For the same mechanistic reason, in certain embodiments, it is anticipated that other acid groups or any other group that can form an ionic bond with chlorhexidine free base can replace carboxylic groups in part or in whole. Non-limiting examples include sulfonic and phosphonic acids.

In certain embodiments, provided are compositions of nonionically stabilized polyurethane dispersions/solutions chemically bonded to chlorhexidine free base via a salt linkage. Quite surprisingly, it was found that chlorhexidine maintained its biocidal properties even though it was immobilized by the polymer matrix through ionic bonding. Such polymeric salt compositions have been found to not only have high antimicrobial functionality, but also retained this functionality through leaching testing, enabling its use in a coating application to provide a surface with long-term antibacterial efficacy. The persistence and durability of antimicrobial properties are important because even biocidal surfaces can be soiled, and harmful microbes can start growing on the top of the dirt and contaminants. These contaminated surfaces need to be washed, and most cleaning solutions are water-based, which would result in leaching of chlorhexidine in conventional systems.

An objective of the present subject matter is to create a useful polymer or polymer dispersion/solution that can be precisely dosed with chlorhexidine in a biologically active form to have controlled resistance to microbial growth. Another objective is to provide a chemical mechanism to retain the chlorhexidine with the polymer during exposure to water or solvents, such that chlorhexidine doesn't need to be re-applied on a too-frequent basis to the polymer to maintain a desired level of microbial growth resistance.

Chlorhexidine digluconate (CHG) is a prevailing form of chlorhexidine in antimicrobial applications. However, CHG tends to leach out of polymer compositions because of its high solubility in water: it is soluble in water to at least 50% (The Merck Index. 12^{th} Edn. page 2136 (1996)). It could be speculated that chlorhexidine cation might be able to migrate from its salt with gluconic acid to the free carboxylic acid of a polyurethane of the present subject matter via the metathesis reaction; however, the acidity of gluconic acid, which may be characterized by its pKa of 3.86, is stronger than that of carboxylic group in polyurethane. The pKa of the latter is estimated to be about 7.3, which means that it is substantially neutral. (“Hydrolytically-stable polyester-polyurethane nanocomposites.” Paper No. 22.5. European Coatings Congress. Mar. 18-19, 2013, Nuremberg, Germany. Alex Lubnin, Gregory R. Brown, Elizabeth A. Flores, Nai Z. Huang, Pamela Izquierdo, Susan L. Lenhard, and Ryan Smith.) This means that the gluconate anion's bond with chlorhexidine cation is stronger, and such a metathesis reaction will not occur.

It was unexpectedly discovered that chlorhexidine free base had sufficient solubility in water to migrate from chlorhexidine-rich phases, through the aqueous phase, and into polyurethane particles and/or molecules having free (non-reacted and non-salted) carboxylic acid groups to form chlorhexidine salts with those carboxylic acid groups. This resulted in polyurethane solutions, dispersions, films, etc., having chlorhexidine present in a substantially non-migrating form that retains its biocidal activity, even though bound to a polymer.

Commercial polyurethane dispersions in water have carboxylic or other acid groups which have been neutralized with a base, such as tertiary amines, NaOH, KOH, or NH₄OH, to impart dispersibility and colloidal anionic stabilization of the polyurethane particles in a water or a polar organic medium. Because acid groups diminish chemical and water resistance and durability of urethanes, an effort is made to minimize their content and fully neutralize them to maximize their dispersing power. As such, these polyurethane dispersions are substantially free of carboxylic acid groups when in the form of polyurethane dispersions in an aqueous medium.

In certain embodiments of the present subject matter, therefore, it is desirable to reduce the amount of base used to neutralize the polyurethane, to leave at least some acid groups free to form a salt bond with the biguanide free base materials described herein. In certain embodiments, a substantial portion of acid in the dispersing monomer is left unneutralized. In certain embodiments, the molar or equivalent ratio of the acid to neutralizing base (such as amine) may be (acid:base): 1:0.95; 1:0.9; 1:0.8; 1:0.7; 1:0.6; 1:0.5; 1:0.4; 1:0.3; 1.02; or 1:0.1. In certain embodiments, the molar amount of neutralizing base relative to the each mole of acid groups in the polyurethane may be from 0.1 to 0.95, from 0.1 to 0.9, from 0.1 to 0.8, from 0.1 to 0.7, from 0.1 to 0.6, from 0.1 to 0.5, from 0.1 to 0.4, from 0.1 to 0.3, from 0.1 to 0.2, from 0.2 to 0.95, from 0.2 to 0.9, from 0.2 to 0.8, from 0.2 to 0.7, from 0.2 to 0.6, from 0.2 to 0.5, from 0.2 to 0.4, from 0.2 to 0.3, from 0.3 to 0.95, from 0.3 to 0.9, from 0.3 to 0.8, from 0.3 to 0.7, from 0.3 to 0.6, from 0.3 to 0.5, from 0.3 to 0.4, from 0.4 to 0.95, from 0.4 to 0.9, from 0.4 to 0.8, from 0.4 to 0.7, from 0.4 to 0.6, from 0.4 to 0.5, from 0.5 to 0.95, from 0.5 to 0.9, from 0.5 to 0.8, from 0.5 to 0.7, from 0.5 to 0.6, from 0.6 to 0.95, from 0.6 to 0.9, from 0.6 to 0.8, from 0.6 to 0.7, from 0.7 to 0.95, from 0.7 to 0.9, from 0.7 to 0.8, from 0.8 to 0.95, from 0.8 to 0.9, or from 0.9 to 0.95.

A feature of the desired prepolymer and polyurethane from the prepolymer of the present subject matter is the presence of what we call poly(alkylene oxide) tethered and/or terminal macromonomer at levels sufficient to make stable urethane dispersion/solution and incorporate monomers with free acid groups without neutralizing them, wherein the alkylene of the alkylene oxide has from 2 to 10 carbon atoms (such as 2 to 4, or 2 to 3 carbon atoms, and optionally wherein at least 80 mole percent of the alkylene oxide repeating units have 2 carbon atoms per repeat unit), wherein the tethered and/or terminal macromonomer is described as a macromonomer having a number average molecular weight of at least 300 g/mole and one or more functional reactive groups characterized as active hydrogen groups (or alternatively characterized as groups reactive with isocyanate groups to form a covalent chemical bond (such as urethane or urea)), the reactive groups (e.g., amine or hydroxyl groups) primarily at one end of the tethered and/or terminal macromonomer, such that the tethered and/or terminal macromonomer has at least one non-reactive end (e.g., non-reactive with isocyanate groups to form a covalent urethane or urea bond), such as just one non-reactive group, and at least 50 wt. % of the alkylene oxide repeat units of the macromonomer are between the non-reactive end of the tethered and/or terminal macromonomer and the closest reactive group of the macromonomer to the non-reactive terminus.

In certain embodiments, a polyurethane composition is provided, comprising a polyurethane with at least one free acid group salted with a biguanide free base.

In certain embodiments, the at least one free acid group comprises at least one of carboxylic acid, sulfonic acid, or phosphonic acid.

In certain embodiments, the biguanide free base comprises a bisbiguanide free base.

In certain embodiments, the biguanide free base comprises at least one of chlorhexidine free base, alexidine free base, polyhexanide free base, or polyaminopropyl biguanide free base.

In certain embodiments, the polyurethane comprises the reaction product of: (a) a polyisocyanate component having on average two or more isocyanate groups; (b) a poly(alkylene oxide) tethered and/or terminal macromonomer, wherein the alkylene of the alkylene oxide has from 2 to 10 carbon atoms, wherein the macromonomer has a number average molecular weight of at least 300 g/mole and one or more functional reactive groups characterized as active hydrogen groups, the reactive groups primarily at one end of the macromonomer, such that the macromonomer has at least one non-reactive end, and at least 50 wt. % of the alkylene oxide repeat units of the macromonomer are between the non-reactive end of the macromonomer and the closest reactive group of the macromonomer to the non-reactive terminus; (c) an isocyanate-reactive compound having at least one free acid group; and (d) optionally at least one active-hydrogen containing compound other than (b) or (c).

In certain embodiments, the polyurethane has from 12 (such as 15, 20, 25, 30, 35, 40, 45, or 50) wt. % to about 80 (such as 75, 70, 65, 60, or 55) wt. % of alkylene oxide units present in the poly(alkylene oxide) macromonomer.

In certain embodiments, the at least one free acid group is salted with a biguanide free base to create an ionic salt bond between the at least one free acid group and the biguanide.

In certain embodiments, the molar ratio of biguanide to the at least one free acid group is from 1.2:1 to 0.1:1, such as from 1.1:1 to 0.1:1, 1:1 to 0.1:1, 0.9:1 to 0.1:1, 0.8:1 to 0.1:1, 0.7:1 to 0.1:1, 0.6:1 to 0.1:1, 0.5:1 to 0.1:1, 0.4:1 to 0.1:1, 0.3:1 to 0.1:1, 0.2:1 to 0.1:1, 1.2:1 to 0.2:1, 1.1:1 to 0.2:1, 1:1 to 0.2:1, 0.9:1 to 0.2:1, 0.8:1 to 0.2:1, 0.7:1 to 0.2:1, 0.6:1 to 0.2:1, 0.5:1 to 0.2:1, 0.4:1 to 0.2:1, 0.3:1 to 0.2:1, 1.2:1 to 0.3:1, 1.1:1 to 0.3:1, 1:1 to 0.3:1, 0.9:1 to 0.3:1, 0.8:1 to 0.3:1, 0.7:1 to 0.3:1, 0.6:1 to 0.3:1, 0.5:1 to 0.3:1, 0.4:1 to 0.3:1, 1.2:1 to 0.4:1, 1.1:1 to 0.4:1, 1:1 to 0.4:1, 0.9:1 to 0.4:1, 0.8:1 to 0.4:1, 0.7:1 to 0.4:1, 0.6:1 to 0.4:1, 0.5:1 to 0.4:1, 1.2:1 to 0.5:1, 1.1:1 to 0.5:1, 1:1 to 0.5:1, 0.9:1 to 0.5:1, 0.8:1 to 0.5:1, 0.7:1 to 0.5:1, 0.6:1 to 0.5:1, 1.2:1 to 0.6:1, 1.1:1 to 0.6:1, 1:1 to 0.6:1, 0.9:1 to 0.6:1, 0.8:1 to 0.6:1, 0.7:1 to 0.6:1, 1.2:1 to 0.7:1, 1.1:1 to 0.7:1, 1:1 to 0.7:1, 0.9:1 to 0.7:1, 0.8:1 to 0.7:1, 1.2:1 to 0.8:1, 1.1:1 to 0.8:1, 1:1 to 0.8:1, 0.9:1 to 0.8:1, 1.2:1 to 0.9:1, 1.1:1 to 0.9:1, 1:1 to 0.9:1, 1.2:1 to 1:1, 1.1:1 to 1:1, or 1.2:1 to 1.1:1.

In certain embodiments, the at least one free acid group is present in the polyurethane at a concentration of from 0.002 (such as 0.003, 0.004, 0.005, 0.006, 0.007, 0.008, 0.009, 0.01, 0.02, 0.03, 0.04, 0.05, 0.06, 0.07, 0.08, 0.09, or 0.1) to 5 (such as 4.5, 4, 3.5, 3, 2.5, 2, 1.5, 1, 0.9, 0.8, 0.7, 0.6, 0.5, 0.4, 0.3, or 0.2) millimoles/gram of polyurethane before being salted with the biguanide free base.

In certain embodiments, the biguanide free base is present in the composition at an amount of from 0.25 (such as 0.3, 0.35, 0.4, 0.45, 0.5, 0.55, 0.6, 0.65, 0.7, 0.75, 0.8, 0.85, 0.9, 0.95, or 1) to 10 (such as 9, 8, 7, 6, 5, 4, 3, or 2) wt. %, based on the total weight of the polyurethane.

In certain embodiments, the polyurethane has from 40 (such as 45, 50, 55, or 60) to 80 (such as 75, 70, or 65) wt. % alkylene oxide repeat units present in repeat units of the macromonomer.

In certain embodiments, the poly(alkylene oxide) chains of the macromonomer have number average molecular weights from about 88 (such as 90, 100, 200, 300, 400, 500, 600, 700, 800, 900, or 1,000) to 10,000 (such as 9,000, 8,000, 7,000, 6,000, 5,000, 4,000, 3,000, or 2,000) g/mole.

In certain embodiments, the poly(alkylene oxide) chains of the macromonomer have at least 50% (such as 55%, 60%, 65%, 70%, 75%, 80%, 85%, 90%, 95%, 99%, or 100%) ethylene oxide units based on their total alkylene oxide units.

In certain embodiments, the polyurethane compositions described herein may be formulated with other polymers, such as polyurethanes not including free acid groups, to form a desirable coating composition depending on the properties desired of a particular coating compositions. Other ingredients may also be added to the compositions to provide desired properties.

In certain embodiments, the present composition may be used as a coating on a surface.

The following embodiments of the present subject matter are contemplated:

1. A polyurethane composition comprising a polyurethane with at least one free acid group salted with a biguanide free base.

2. The composition of embodiment 1, wherein the at least one free acid group comprises at least one of carboxylic acid, sulfonic acid, or phosphonic acid.

3. The composition of either embodiment 1 or embodiment 2, wherein the biguanide free base comprises a bisbiguanide free base.

4. The composition of any one of embodiments 1 to 3, wherein the biguanide free base comprises at least one of chlorhexidine free base, alexidine free base, polyhexanide free base, or polyaminopropyl biguanide free base.

5. The composition of any one of embodiments 1 to 4, wherein the polyurethane comprises the reaction product of: (a) a polyisocyanate component having on average two or more isocyanate groups; (b) a poly(alkylene oxide) tethered and/or terminal macromonomer, wherein the alkylene of the alkylene oxide has from 2 to 10 carbon atoms, wherein the macromonomer has a number average molecular weight of at least 300 g/mole and one or more functional reactive groups characterized as active hydrogen groups, the reactive groups primarily at one end of the macromonomer, such that the macromonomer has at least one non-reactive end, and at least 50 wt. % of the alkylene oxide repeat units of the macromonomer are between the non-reactive end of the macromonomer and the closest reactive group of the macromonomer to the non-reactive terminus; (c) an isocyanate-reactive compound having at least one free acid group; and (d) optionally at least one active-hydrogen containing compound other than (b) or (c).

6. The composition of any one of embodiments 1 to 5, wherein the polyurethane has from 12 wt. % to about 80 wt. % of alkylene oxide units present in the poly(alkylene oxide) macromonomer.

7. The composition of any one of embodiments 1 to 6, wherein the at least one free acid group is salted with a biguanide free base to create an ionic salt bond between the at least one free acid group and the biguanide.

8. The composition of any one of embodiments 1 to 7, wherein the molar ratio of biguanide to the at least one free acid group is from 1.2:1 to 0.1:1.

9. The composition of any one of embodiments 1 to 8, wherein the at least one free acid group is present in the polyurethane at a concentration of from 0.002 to 5 millimoles/gram of polyurethane before being salted with the biguanide free base.

10. The composition of any one of embodiments 1 to 9, wherein the biguanide free base is present in the composition at an amount of from 0.25 to 10 wt. %, based on the total weight of the polyurethane.

11. The composition of any one of embodiments 1 to 10, wherein the polyurethane has from 40 to 80 wt. % alkylene oxide repeat units present in repeat units of the macromonomer.

12. The composition of any one of embodiments 1 to 11, wherein the poly(alkylene oxide) chains of the macromonomer have number average molecular weights from about 88 to 10,000 g/mole.

13. The composition of any one of embodiments 1 to 12, wherein the poly(alkylene oxide) chains of the macromonomer have at least 50% ethylene oxide units based on their total alkylene oxide units.

14. A coating comprising the composition of any one of embodiments 1 to 13, used as a coating on a surface.

### DETAILED DESCRIPTION

Various features and embodiments of the present subject matter will be described below by way of non-limiting illustration.

As used herein, the indefinite article “a”/“an” is intended to mean one or more than one. As used herein, the phrase “at least one” means one or more than one of the following term(s). Thus, “a”/“an” and “at least one” may be used interchangeably. For example, “at least one of A, B or C” means that just one of A, B or C may be included, and any mixture of two or more of A, B and C may be included, in alternative embodiments. As another example, “at least one X” means that one or more than one material/component X may be included.

As used herein, the term “about” means that a value of a given quantity is within ±20% of the stated value. In other embodiments, the value is within ±15% of the stated value. In other embodiments, the value is within ±10% of the stated value. In other embodiments, the value is within ±5% of the stated value. In other embodiments, the value is within ±2.5% of the stated value. In other embodiments, the value is within ±1% of the stated value. In other embodiments, the value is within a range of the explicitly-described value which would be understood by those of ordinary skill, based on the disclosures provided herein, to perform substantially similarly to compositions including the literal amounts described herein.

As used herein, the term “substantially” means that a value of a given quantity is within ±10% of the stated value. In other embodiments, the value is within ±5% of the stated value. In other embodiments, the value is within ±2.5% of the stated value. In other embodiments, the value is within ±1% of the stated value.

As used herein, the transitional term “comprising,” which is synonymous with “including,” “containing,” or “characterized by,” is inclusive or open-ended and does not exclude additional, un-recited elements or method steps. However, in each recitation of “comprising” herein, it is intended that the term also encompass, as alternative embodiments, the phrases “consisting essentially of” and “consisting of,” where “consisting of” excludes any element or step not specified and “consisting essentially of” permits the inclusion of additional un-recited elements or steps that do not materially affect the essential or basic and novel characteristics of the composition or method under consideration.

There is provided a polyurethane solution and/or dispersion in an aqueous medium that is stabilized (e.g., colloidally stabilized if a dispersion) with poly(alkylene oxide) tethered and/or terminal macromonomer(s) such that the poly(alkylene oxide) of the tethered and/or terminal macromonomer extends from the polyurethane into the aqueous phase and provides (colloidal) stabilization or dissolution of the polyurethane and/or polyurethane particles. The polyurethane particles can also have anionic stabilization from the incorporation of acid-containing molecules (such as carboxylic acid-containing molecules incorporated into the polyurethane). Depending on whether the carboxylic acid groups are salted or non-salted, they may function to provide colloidal stabilization or reaction sites to bind chlorhexidine free base during a salting reaction for the polyurethane. At least a portion of the carboxylic acid groups must remain in the free acid form after the polyurethane synthesis, such that they are available to salt with the chlorhexidine free base.

When coating manufacturers wanted to create polyurethane coatings that were low in volatile organic solvents, they created polyurethane dispersions in aqueous media. The first polyurethane dispersions were anionically stabilized with acid groups that were salted with bases to create ionic groups that colloidally stabilized the dispersion. Later coating manufacturers developed nonionic poly(alkylene oxide) (such as poly(ethylene oxide)) macromonomers that could be reacted onto/into polyurethane prepolymers and provide a potential alternative or supplement to anionic colloidal stabilization. Anionic colloidal stabilization was subject to destabilization from cations and salts. Nonionic colloidal stabilization was resistant to destabilization.

The present subject matter relates to polyurethanes salted with chlorhexidine, and its preparation is exemplified by a sip-called “prepolymer process” comprising: (A) reacting to form an isocyanate-terminated prepolymer: (1) at least one polyisocyanate having an average of about two or more isocyanate groups; (2) at least one poly(alkylene oxide) tethered and/or terminal macromonomer(s), wherein the alkylene of the alkylene oxide has from 2 to 10 carbon atoms (such as 2 to 4, or 2 to 3 carbon atoms, and optionally wherein at least 80 mole percent of the alkylene oxide repeating units have 2 carbon atoms per repeat unit), wherein the tethered and/or terminal macromonomer is described as a macromonomer having a number average molecular weight of at least 300 g/mole and one or more functional reactive groups characterized as active hydrogen groups or characterized as groups reactive with isocyanate groups to form a covalent chemical bond (such as urethane or urea), the reactive groups (e.g., amine or hydroxyl groups) primarily at one end of the tethered and/or terminal macromonomer, such that the tethered and/or terminal macromonomer has at least one non-reactive end (non-reactive with isocyanate groups to form a covalent urethane or urea bond), such as just one non-reactive group, and at least 50 wt. % of the alkylene oxide repeat units of the macromonomer are between the non-reactive end of the tethered and/or terminal macromonomer and the closest reactive group of the macromonomer to the non-reactive terminus; (3) at least one compound having at least one carboxylic acid functional group; and (4) optionally at least one other active hydrogen-containing compound other than (2) and (3), in order to form an isocyanate-terminated prepolymer; (B) dissolving and/or dispersing the prepolymer in water, and chain extending the prepolymer by reaction with at least one of water, inorganic or organic polyamine having an average of about 2 or more primary and/or secondary amine groups, polyols, or combinations thereof; and (C) thereafter further processing the chain-extended solution and/or dispersion of step (B) in order to form a composition or article with the ability to salt with chlorhexidine.

It is noted that other processes, well known to those skilled in the art, can also be used to manufacture the salt-able polyurethanes of the present subject matter if they use the required amount of acid-containing monomer in a free acid form, including but not limited to the following: dispersing prepolymer by shear forces with emulsifiers; the so-called “acetone process”; melt dispersion processes; ketazine and ketamine processes; non-isocyanate processes; continuous processes; reverse feed processes; solution polymerization; bulk polymerization; and reactive extrusion processes.

In certain embodiments, it may be desirable to utilize poly(ethylene oxide) monomers as the poly(alkylene oxide) content of the polyurethanes disclosed herein. All possible poly(ethylene oxide) monomers, which can be used in polyurethane synthesis, may be divided into three families: tethered, terminal and main-chain. Tethered (or side-chain) and terminal monomers have at least one chain end that is unreactive in the polyurethane synthesis and at least one chain end that has at least one group that is reactive in the polyurethane synthesis and can participate in the polymer building. They can be represented by the following general formula:

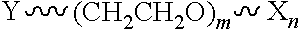

where Y is any unreactive group, X is any reactive group such as alcohol, amine, mercaptan, isocyanate, etc., n=1, 2, or 3, and m=1 and more. These include branched structures and copolymers with other alkylene oxides such as propylene oxide. Examples of the tethered monomers are Tegomer® D-3403 from Evonik Industries and Ymer™ N120 from Perstorp, which have the following formula:

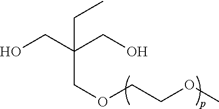

wherein p is the number of ethylene oxide units or degree of polymerization.

Examples of the terminal monomers are the so-called MPEGs (monomethyl ether of polyethyleneglycol) which have the following formula:

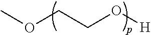

wherein p is the number of ethylene oxide units or degree of polymerization.

Main-chain poly(ethylene oxide) monomers have at least two chain ends that are reactive in the polyurethane synthesis. This family can be represented by the following general formula:

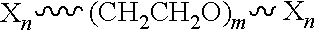

where X is any reactive group such as alcohol, amine, mercaptan, isocyanate, etc., n=1, 2, or 3, and m=1 and more. For example:

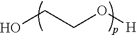

wherein p is the number of ethylene oxide units or degree of polymerization.

In certain embodiments, it may be desirable to control the amount of tethered, terminal, and/or main-chain ethylene oxide groups present in the polyurethanes disclosed herein, as doing so may provide desirable properties.

It is to be understood that the ethylene oxide monomeric unit content of the polyurethanes disclosed herein may be present in the main chain of the polyurethane, the side chain(s) of the polyurethane (i.e., tethered groups), and/or in terminal groups of the polyurethane. The relative amounts of ethylene oxide monomeric units present in each of these portions of the polyurethane molecule(s) may impact the properties of the polyurethane. The embodiments described herein which refer to the amounts of ethylene oxide monomeric units should be considered to be combinable with each other, to the extent that doing so is physically possible.

In certain embodiments, the polyurethane comprises ethylene oxide monomeric side-chain units in an amount of 12% (such as 15%, 20%, 25%, 30%, 35%, 40%, 45% or 50%) to 80% (such as 75%, 70%, 65%, 60%, or 55%) by weight, based on the total dry weight of the polyurethane.

In certain embodiments, the polyurethane comprises ethylene oxide monomeric main-chain units in an amount of less than 75% (such as 70%, 65%, 60%, 55%, 50%, 45%, 40%, 35%, 30%, 25%, 20%, 15%, 10%, 5%, 4%, 3%, 2%, or 1%) by weight, based on the total dry weight of the polyurethane. In certain embodiments, the polyurethane is substantially free of ethylene oxide monomeric main-chain units. In certain embodiments, the polyurethane is free of ethylene oxide monomeric main-chain units.

In certain embodiments, 100% of all ethylene oxide monomeric units in the polyurethane comprise ethylene oxide monomeric side-chain units and/or poly(ethylene oxide) terminal groups. In certain embodiments, 100% of all ethylene oxide monomeric units in the polyurethane comprise ethylene oxide monomeric side-chain units. In certain embodiments, 100% of all ethylene oxide monomeric units in the polyurethane comprise poly(ethylene oxide) terminal groups.

In certain embodiments, at least 95% of all ethylene oxide monomeric units in the polyurethane comprise ethylene oxide monomeric side-chain units and/or poly(ethylene oxide) terminal groups. In certain embodiments, at least 95% of all ethylene oxide monomeric units in the polyurethane comprise ethylene oxide monomeric side-chain units. In certain embodiments, at least 95% of all ethylene oxide monomeric units in the polyurethane comprise poly(ethylene oxide) terminal groups.

In certain embodiments, at least 90% of all ethylene oxide monomeric units in the polyurethane comprise ethylene oxide monomeric side-chain units and/or poly(ethylene oxide) terminal groups. In certain embodiments, at least 90% of all ethylene oxide monomeric units in the polyurethane comprise ethylene oxide monomeric side-chain units. In certain embodiments, at least 90% of all ethylene oxide monomeric units in the polyurethane comprise poly(ethylene oxide) terminal groups.

In certain embodiments, at least 85% of all ethylene oxide monomeric units in the polyurethane comprise ethylene oxide monomeric side-chain units and/or poly(ethylene oxide) terminal groups. In certain embodiments, at least 85% of all ethylene oxide monomeric units in the polyurethane comprise ethylene oxide monomeric side-chain units. In certain embodiments, at least 85% of all ethylene oxide monomeric units in the polyurethane comprise poly(ethylene oxide) terminal groups.

In certain embodiments, at least 80% of all ethylene oxide monomeric units in the polyurethane comprise ethylene oxide monomeric side-chain units and/or poly(ethylene oxide) terminal groups. In certain embodiments, at least 80% of all ethylene oxide monomeric units in the polyurethane comprise ethylene oxide monomeric side-chain units. In certain embodiments, at least 80% of all ethylene oxide monomeric units in the polyurethane comprise poly(ethylene oxide) terminal groups.

In certain embodiments, at least 75% of all ethylene oxide monomeric units in the polyurethane comprise ethylene oxide monomeric side-chain units and/or poly(ethylene oxide) terminal groups. In certain embodiments, at least 75% of all ethylene oxide monomeric units in the polyurethane comprise ethylene oxide monomeric side-chain units. In certain embodiments, at least 75% of all ethylene oxide monomeric units in the polyurethane comprise poly(ethylene oxide) terminal groups.

In certain embodiments, at least 70% of all ethylene oxide monomeric units in the polyurethane comprise ethylene oxide monomeric side-chain units and/or poly(ethylene oxide) terminal groups. In certain embodiments, at least 70% of all ethylene oxide monomeric units in the polyurethane comprise ethylene oxide monomeric side-chain units. In certain embodiments, at least 70% of all ethylene oxide monomeric units in the polyurethane comprise poly(ethylene oxide) terminal groups.

In certain embodiments, at least 65% of all ethylene oxide monomeric units in the polyurethane comprise ethylene oxide monomeric side-chain units and/or poly(ethylene oxide) terminal groups. In certain embodiments, at least 65% of all ethylene oxide monomeric units in the polyurethane comprise ethylene oxide monomeric side-chain units. In certain embodiments, at least 65% of all ethylene oxide monomeric units in the polyurethane comprise poly(ethylene oxide) terminal groups.

In certain embodiments, at least 60% of all ethylene oxide monomeric units in the polyurethane comprise ethylene oxide monomeric side-chain units and/or poly(ethylene oxide) terminal groups. In certain embodiments, at least 60% of all ethylene oxide monomeric units in the polyurethane comprise ethylene oxide monomeric side-chain units. In certain embodiments, at least 60% of all ethylene oxide monomeric units in the polyurethane comprise poly(ethylene oxide) terminal groups.

In certain embodiments, at least 55% of all ethylene oxide monomeric units in the polyurethane comprise ethylene oxide monomeric side-chain units and/or poly(ethylene oxide) terminal groups. In certain embodiments, at least 55% of all ethylene oxide monomeric units in the polyurethane comprise ethylene oxide monomeric side-chain units. In certain embodiments, at least 55% of all ethylene oxide monomeric units in the polyurethane comprise poly(ethylene oxide) terminal groups.

In certain embodiments, at least 50% of all ethylene oxide monomeric units in the polyurethane comprise ethylene oxide monomeric side-chain units and/or poly(ethylene oxide) terminal groups. In certain embodiments, at least 50% of all ethylene oxide monomeric units in the polyurethane comprise ethylene oxide monomeric side-chain units. In certain embodiments, at least 50% of all ethylene oxide monomeric units in the polyurethane comprise poly(ethylene oxide) terminal groups.

In certain embodiments, at least 45% of all ethylene oxide monomeric units in the polyurethane comprise ethylene oxide monomeric side-chain units and/or poly(ethylene oxide) terminal groups. In certain embodiments, at least 45% of all ethylene oxide monomeric units in the polyurethane comprise ethylene oxide monomeric side-chain units. In certain embodiments, at least 45% of all ethylene oxide monomeric units in the polyurethane comprise poly(ethylene oxide) terminal groups.

In certain embodiments, at least 40% of all ethylene oxide monomeric units in the polyurethane comprise ethylene oxide monomeric side-chain units and/or poly(ethylene oxide) terminal groups. In certain embodiments, at least 40% of all ethylene oxide monomeric units in the polyurethane comprise ethylene oxide monomeric side-chain units. In certain embodiments, at least 40% of all ethylene oxide monomeric units in the polyurethane comprise poly(ethylene oxide) terminal groups.

In certain embodiments, at least 35% of all ethylene oxide monomeric units in the polyurethane comprise ethylene oxide monomeric side-chain units and/or poly(ethylene oxide) terminal groups. In certain embodiments, at least 35% of all ethylene oxide monomeric units in the polyurethane comprise ethylene oxide monomeric side-chain units. In certain embodiments, at least 35% of all ethylene oxide monomeric units in the polyurethane comprise poly(ethylene oxide) terminal groups.

In certain embodiments, at least 30% of all ethylene oxide monomeric units in the polyurethane comprise ethylene oxide monomeric side-chain units and/or poly(ethylene oxide) terminal groups. In certain embodiments, at least 30% of all ethylene oxide monomeric units in the polyurethane comprise ethylene oxide monomeric side-chain units. In certain embodiments, at least 30% of all ethylene oxide monomeric units in the polyurethane comprise poly(ethylene oxide) terminal groups.

In certain embodiments, at least 25% of all ethylene oxide monomeric units in the polyurethane comprise ethylene oxide monomeric side-chain units and/or poly(ethylene oxide) terminal groups. In certain embodiments, at least 25% of all ethylene oxide monomeric units in the polyurethane comprise ethylene oxide monomeric side-chain units. In certain embodiments, at least 25% of all ethylene oxide monomeric units in the polyurethane comprise poly(ethylene oxide) terminal groups.

Adjusting the ethylene oxide monomeric unit content of the polyurethane may modulate the hydrophilic characteristics of the polyurethane. For example, an ethylene oxide monomeric unit content of at least about 20% (such as not less than 50%) by weight, based on the total weight of the polyurethane, may render the polyurethane soluble in water. For example, the polyurethane may comprise from 35% to 90% by weight ethylene oxide monomeric units, based on the total weight of the polyurethane. Furthermore, polyurethanes having ethylene oxide side-chain units in an amount of 12% to 80% by weight, based on the total weight of the polyurethane, may be desirable for certain applications. In certain embodiments, it may be desirable to limit the amount of ethylene oxide main-chain units to an amount of less than 25% by weight, based on the total weight of the polyurethane. In certain embodiments, polyethylene oxide side chains may be desirable, in that they may prevent the polyurethane from swelling to an undesirable degree in water, which may cause undesirably high viscosity.

The compositions of the present subject matter are conveniently referred to as polyurethanes because they contain urethane groups. The prepolymers and polymers can be more accurately described as poly(urethane/urea)s if the active hydrogen-containing compounds are polyols and/or polyamines. It is well understood by those skilled in the art that “polyurethanes” is a generic term used to describe polymers obtained by reacting isocyanates with at least one hydroxyl-containing compound, amine-containing compound, or mixture thereof. It also is well understood by those skilled in the art that polyurethanes may also include allophanate, biuret, carbodiimide, oxazolidinyl, isocyanurate, uretdione, and other linkages in addition to urethane and urea linkages.

As used herein, the term “wt. %” means the number of parts by weight of monomer per 100 parts by weight of polymer on a dry weight basis, or the number of parts by weight of ingredient per 100 parts by weight of specified composition. As used herein, the term “molecular weight” means number average molecular weight.

### Polyisocyanates

Suitable polyisocyanates have an average of about two or more isocyanate groups, such as an average of about two to about four isocyanate groups, optionally an average of two isocyanate groups, and include aliphatic, cycloaliphatic, araliphatic, and aromatic polyisocyanates, used alone or in mixtures of two or more.

Specific examples of suitable aliphatic polyisocyanates include alpha, omega-alkylene diisocyanates having from 5 to 20 carbon atoms, such as hexamethylene-1,6-diisocyanate, 1,12-dodecane diisocyanate, 2,2,4-trimethyl-hexamethylene diisocyanate, 2,4,4-trimethyl-hexamethylene diisocyanate, 2-methyl-1,5-pentamethylene diisocyanate, and the like. Polyisocyanates having fewer than 5 carbon atoms can be used but may be unsuitable in certain embodiments because of their high volatility and toxicity. Exemplary aliphatic polyisocyanates include hexamethylene-1,6-diisocyanate, 2,2,4-trimethyl-hexamethylene-diisocyanate, and 2,4,4-trimethyl-hexamethylene diisocyanate.

Specific examples of suitable cycloaliphatic polyisocyanates include dicyclohexylmethane diisocyanate, isophorone diisocyanate, 1,4-cyclohexane diisocyanate, 1,3-bis-(isocyanatomethyl) cyclohexane, and the like. Suitable cycloaliphatic polyisocyanates include dicyclohexylmethane diisocyanate and isophorone diisocyanate.

Specific examples of suitable araliphatic polyisocyanates include m-tetramethyl xylylene diisocyanate, p-tetramethyl xylylene diisocyanate, 1,4-xylylene diisocyanate, 1,3-xylylene diisocyanate, and the like. A suitable araliphatic polyisocyanate is tetramethyl xylylene diisocyanate.

Examples of suitable aromatic polyisocyanates include 4,4′-diphenylmethylene diisocyanate), toluene diisocyanate, their isomers, naphthalene diisocyanate, and the like. A suitable aromatic polyisocyanate is toluene diisocyanate. Polyisocyanates having three or more isocyanate groups (or dimers or trimers of diisocyanates) can be used in this embodiment, especially when the prepolymer is partially or fully made with poly(alkylene oxide) oligomer/chains (one option for the poly(alkylene oxide) tethered and/or terminal macromonomer) with only one active hydrogen group capable of reacting with an isocyanate group at one end of the poly(alkylene oxide) and the other (at least one end) of the poly(alkylene oxide) being non-reactive with isocyanate groups.

### Active Hydrogen-Containing Compounds

The term “active hydrogen-containing” refers to compounds that are a source of active hydrogen and that can react with isocyanate groups, such as via the following reaction: —NCO+H—X-->NH—C(—O)—X. The active hydrogen containing compounds include both the poly(alkylene oxide) tethered and/or terminal macromonomer and the other active hydrogen compound that is other than the poly(alkylene oxide) tethered and/or terminal macromonomer. Examples of suitable active hydrogen-containing compounds include but are not limited to polyols, polythiols and polyamines.

As used herein, the term “alkylene oxide” includes both alkylene oxides and substituted alkylene oxides having 2 or more carbon atoms, such as 2 to 10 carbon atoms. The active hydrogen-containing compounds used in this disclosure have poly(alkylene oxide) tethered and/or terminal macromonomer sufficient in amount such that the poly(alkylene oxide) of the tethered and/or terminal macromonomer comprises about 12 wt. % to about 80 wt, %, such as about 15 wt. % to about 60 wt. %, or about 20 wt. % to about 50 wt. %, of poly(alkylene oxide) units in the final polyurethane on a dry weight basis. At least about 50 wt. %, such as at least about 70 wt. %, or at least about 90 wt. %, of the alkylene oxide repeat units of the tethered and/or terminal macromonomer comprise poly(ethylene oxide), and the remainder of the alkylene oxide repeat units can comprise alkylene oxide and substituted alkylene oxide units having from 3 to about 10 carbon atoms, such as propylene oxide, tetramethylene oxide, butylene oxides, epichlorohydrin, epibromohydrin, allyl glycidyl ether, styrene oxide, and the like, and mixtures thereof. The term “final polyurethane” means the polyurethane produced after formation of the prepolymer followed by the chain extension step as described more fully herein.

Such active hydrogen-containing compounds provide less than about 25 wt. %, such as less than about 15 wt. %, or less than about 5 wt. %, poly(ethylene oxide) units in the backbone (main chain) based upon the dry weight of final polyurethane, since such main-chain poly(ethylene oxide) units tend to cause swelling of polyurethane particles in the waterborne polyurethane dispersion and may also contribute to lower in-use tensile strength of articles made from the polyurethane dispersion. Mixtures of active hydrogen-containing compounds having poly(alkylene oxide) tethered and/or terminal chains can be used with active hydrogen-containing compounds not having such tethered and/or terminal chains.

The polyurethanes of the present subject natter may also have reacted therein at least one active hydrogen-containing compound not having the poly(alkylene oxide) tethered and/or terminal macromonomer chains, perhaps ranging widely in molecular weight from about 88 to about 10,000 grams/mole, such as about 200 to about 6,000 grams/mole, or about 300 to about 3,000 grams/mole. Suitable active-hydrogen containing compounds not having the side chains include any of the amities and polyols described herein.

The term “polyol” denotes any compound having an average of about two or more hydroxyl groups per molecule. Examples of such polyols that can be used in the present subject matter include polymeric polyols such as polyester polyols and polyether polyols, as well as polyhydroxy polyester amides, hydroxyl-containing polycaprolactones, hydroxyl-containing epoxides, polyhydroxy polycarbonates, polyhydroxy polyacetals, polyhydroxy polythioethers, polysiloxane polyols, ethoxylated polysiloxane polyols, polybutadiene polyols and hydrogenated polybutadiene polyols, halogenated polyesters and polyethers, and the like, and mixtures thereof. Polyester polyols, polyether polyols, polycarbonate polyols, polysiloxane polyols, and ethoxylated polysiloxane polyols are suitable examples.

Poly(alkylene oxide) tethered and/or terminal chains can be incorporated into such polyols by methods well known to those skilled in the art. For example, active hydrogen-containing compounds having poly(alkylene oxide) tethered and/or terminal (side or terminal) chains include diols having poly(ethylene oxide) side chains such as those described in U.S. Pat. No. 3,905,929 (incorporated herein by reference in its entirety). Further, U.S. Pat. No. 5,700,867 (incorporated herein by reference in its entirety) teaches methods for incorporation of poly(ethylene oxide) side chains at col. 4, line 35 to col. 5, line 45. A suitable active hydrogen-containing compound having poly(ethylene oxide) side chains is Tegomer® D-3403 from Evonik Industries and Ymer™ N120 from Perstorp.

The polyester polyols (which may be difunctional and used as backbone polyurethane units) may be esterification products prepared by the reaction of organic polycarboxylic acids or their anhydrides with a stoichiometric excess of a diol. Examples of suitable polyols for use in the reaction include poly(glycol adipate)s, poly(ethylene terephthalate) polyols, polycaprolactone polyols, orthophthalic polyols, sulfonated and phosphorated polyols, and the like, and mixtures thereof.

The diols used in making the polyester polyols may include alkylene glycols, e.g., ethylene glycol, 1,2- and 1,3-propylene glycols, 1,2-, 1,3-, 1,4-, and 2,3-butylene glycols, hexane diols, neopentyl glycol, 1,6-hexanediol, 1,8-octanediol, and other glycols such as bisphenol-A, cyclohexane diol, cyclohexane dimethanol (1,4-bis-hydroxymethylcycohexane), 2-methyl-1,3-propanediol, 2,2,4-trimethyl-1,3-pentanediol, diethylene glycol, triethylene glycol, tetraethylene glycol, polyethylene glycol, dipropylene glycol, polypropylene glycol, dibutylene glycol, polybutylene glycol, dimerate diol, hydroxylated bisphenols, polyether glycols, halogenated diols, and the like, and mixtures thereof. Suitable diols include ethylene glycol, diethylene butylene glycol, hexane diol, and neopentyl glycol.

Suitable carboxylic acids used in making the polyester polyols include dicarboxylic acids and tricarboxylic acids and anhydrides, e.g., maleic acid, maleic anhydride, succinic acid, glutaric acid, glutaric anhydride, adipic acid, suberic acid, pimelic acid, azelaic acid, sebacic acid, chlorendic acid, 1,2,4-butane-tricarboxylic acid, phthalic acid, isomers of phthalic acid, phthalic anhydride, fumaric acid, dimeric fatty acids such as oleic acid, and the like, and mixtures thereof. Suitable polycarboxylic acids used in making the polyester polyols include aliphatic or aromatic dibasic acids.

A suitable polyester polyol is a diol. Suitable polyester diols include poly(butanediol adipate); copolymers of hexane diol, adipic acid and isophthalic acid; polyesters such as hexane-adipate-isophthalate polyester; hexane diol-neopentyl glycol-adipic acid polyester diols, e.g., Piothane® 67-3000 HNA (Panolam Industries) and Piothane 67-1000 HNA; propylene glycol-maleic anhydride-adipic acid polyester diols, e.g., Piothane 50-1000 PMA; and/or hexane diol-neopentyl glycol-fumaric acid polyester diols, e.g., Piothane 67-500 HNF. Other suitable polyester diols include Rucoflex™ S1015-35, S1040-35, and S-1040-110 (Bayer Corporation).

Polyether diols may be substituted in whole or in part for the polyester diols. Polyether polyols are obtained in known manner by the reaction of (A) the starting compounds that contain reactive hydrogen atoms, such as water or the diols set forth for preparing the polyester polyols, and (B) alkylene oxides, such as ethylene oxide, propylene oxide, butylene oxide, styrene oxide, tetrahydrofuran, epichlorohydrin, and the like, and mixtures thereof. Suitable polyethers include polypropylene glycol), polytetrahydrofuran, and copolymers of poly(ethylene glycol) and poly propylene glycol).

Polycarbonate diols and polyols include those obtained from the reaction of (A) diols, such as 1,3-propanediol, 1,4-butanediol, 1,5-pentanediol, 3-methyl-1,5-pentanediol, 1,6-hexanediol, diethylene glycol, triethylene glycol, tetraethylene glycol, and the like, and mixtures thereof with (B) dialkylcarbonates, diarylcarbonates, or phosgene.

Polyacetals include the compounds that can be prepared from the reaction of (A) aldehydes, such as formaldehyde and the like, and (B) glycols, such as diethylene glycol, triethylene glycol, ethoxylated 4,4′-dihydroxy-diphenyldimethylmethane, 1,6-hexanediol, and the like. Polyacetals can also be prepared by the polymerization of cyclic acetals.

The dials and polyols useful in making polyester polyols can also be used as additional reactants to prepare the isocyanate terminated prepolymer. Instead of a long-chain polyol, a long-chain amine may also be used to prepare the isocyanate-terminated prepolymer. Suitable long-chain amines include polyester amides and polyamides, such as the predominantly linear condensates obtained from reaction of (A) polybasic saturated and unsaturated carboxylic acids or their anhydrides, and (B) polyvalent saturated or unsaturated aminoalcohols, diamines, polyamines, and the like, and mixtures thereof.

Diamines and polyamines are among suitable compounds useful in preparing the polyester amides and polyamides. Suitable diamines and polyamines include 1,2-diaminoethane, 1,6-diaminohexane, 2-methyl-1,5-pentanediamine, 2,2,4-trimethyl-1,6-hexanediamine, 1,12-diaminododecane, 2-aminoethanol, 2-[(2-aminoethyl)amino]-ethanol, piperazine, 2,5-dimethylpiperazine, 1-amino-3-aminomethyl-3,5,5-trimethylcyclohexane (isophorone diamine or IPDA), bis-aminocyclohexyl)-methane, bis-(4-amino-3-methyl-cyclohexyl)-methane, 1,4-diaminocyclohexane, 1,2-propylenediamine, hydrazine, amino acid hydrazides, hydrazides of semicarbazidocarboxylic acids, bis-hydrazides and bis-semicarbazides, diethylene triamine, triethylene tetramine, tetraethylene pentamine, pentaethylene hexamine, N,N,N-tris-(2-aminoethyl)amine, N-(2-piperazinoethyl)-ethylene diamine, N,N′-bis-(2-aminoethyl)-piperazine, N,N,N-tris-(2-aminoethyl)ethylene diamine, N4N-(2-aminoethyl)-2-aminoethyl-N′-(2-aminoethyl)-piperazine, N-(2-aminoethyl)-N-(2-piperazinoethyl)-ethylene diamine, N,N-bis-(2-aminoethyl)-N-(2-piperazinoethyl)amine, N,N-bis-(2-piperazinoethyl)-amine, polyethylene imines, iminobispropylamine, guanidine, melamine, N-(2-aminoethyl)-1,3-propane diamine, 3,3′-diaminobenzidine, 2,4,6-triaminopyrimidine, polyoxypropylene amines, tetrapropylenepentamine, tripropylenetetramine, N,N-bis-(6-aminohexyl)amine, N,N-bis-(3-aminopropyl)ethylene diamine, and 2,4-bis-(4′-aminobenzyl)-aniline, and the like, and mixtures thereof. Suitable diamines and polyamines include 1-amino-3-aminomethyl-3,5,5-trimethylcyclohexane (isophorone diamine or IPDA), bis-(4-aminocyclohexyl)-methane, bis-(4-amino-3-methylcyclohexyl)-methane, ethylene diamine, diethylene triamine, triethylene tetramine, tetraethylene pentamine, and pentaethylene hexamine, and the like, and mixtures thereof. Other suitable diamines and polyamines include Jeffamine™ D-2000 and D-4000, which are amine-terminated polypropylene glycols, differing only by molecular weight, and which are available from Huntsman Chemical Company.

### Prepolymer Ratios of Isocyanate to Active Hydrogen

The ratio of isocyanate to active hydrogen in the prepolymer may range from about 1:1 to about 2.5:1, such as from about 1.3:1 to about 2.5:1, from about 1.5:1 to about 2.1:1, or from about 1.7:1 to about 2:1,

### Compounds Having at Least One Carboxylic Acid Functional Group

Compounds having at least one carboxylic acid functional group include those having one, two or three carboxylic acid groups. A suitable amount of such carboxylic acid compound is up to about 1 milliequivalent, such as from about 0.05 to about 0.5 milliequivalent, or from about 0.1 to about 0.3 milliequivalent, per gram of final polyurethane, on a dry weight basis.

Suitable exemplary monomers with carboxylic acid for incorporation into the isocyanate-terminated prepolymer are hydroxy-carboxylic acids having the general formula (HO)ₓQ(COOH)_{y}, wherein Q is a straight or branched hydrocarbon radical having 1 to 12 carbon atoms, and x and y are each independently 1 to 3. Examples of such hydroxy-carboxylic acids include citric acid, dimethylolpropanoic acid, dimethylol butanoic acid, glycolic acid, lactic acid, malic acid, dihydroxymalic acid, tartaric acid, hydroxypivalic acid, and the like, and mixtures thereof. Dihydroxy-carboxylic acids, such as dimethylolpropanoic acid, are suitable.

Other suitable compounds providing carboxylic acid functionality include thioglycolic acid, 2,6-dihydroxybenzoic acid, and the like, and mixtures thereof.

### Chain Extenders

As a chain extender for the prepolymer, at least one of water, inorganic or organic polyamine having an average of about 2 or more primary and/or secondary amine groups, polyols, or combinations thereof is suitable for use in the present subject matter. Suitable organic amines for use as a chain extender include diethylene triamine (DETA), ethylene diamine (EDA), meta-xylylenediamine (MXDA), aminoethyl ethanolamine (AEEA), 2-methyl pentane diamine, and the like, and mixtures thereof. Also suitable for practice in the present subject matter are propylene diamine, butylene diamine, hexamethylene diamine, cyclohexylene diamine, phenylene diamine, tolylene diamine, 3,3-dichlorobenzidene, 4,4′-methylene-bis-(2-chloroaniline), 3,3-dichloro-4,4-diamino diphenylmethane, sulfonated primary and/or secondary amines, and the like, and mixtures thereof. Suitable inorganic amines include hydrazine, substituted hydrazines, and hydrazine reaction products, and the like, and mixtures thereof. Suitable polyols include those having from 2 to 12 carbon atoms, such as from 2 to 8 carbon atoms, such as ethylene glycol, diethylene glycol, neopentyl glycol, butanediols, hexanediol, and the like, and mixtures thereof. Hydrazine is suitable, such as when used as a solution in water. The amount of chain extender may range from about 0.5 to about 0.95 equivalents based on available isocyanate.

### Polymer Branching

A degree of branching of the prepolymer and/or polyurethane is caused by the desire to have many poly(alkylene oxide) tethered and/or terminal chains with high polyethylene oxide) content extending from the polyurethane central portion of the prepolymer and polyurethane. This degree of branching may be accomplished during the prepolymer step or the extension step. For branching during the extension step, the chain extender DETA (diethylene triamine) is suitable, but other amines having an average of about two or more primary and/or secondary amine groups may also be used. For branching during the prepolymer step, trimethylol propane (IMP) and other polyols having an average of about two or more hydroxyl groups may be used. The branching monomers can be used in any amount. The poly(alkylene oxide) tethered and/or terminal macromonomer will not be considered a branching monomer but does have tethered side chains of poly(alkylene oxide). Also, for branching during the prepolymer step a trifunctional or higher functionality isocyanate may be used.

### Optional Polymer Partial Neutralization

The polyurethanes of the present subject matter can be optionally partially neutralized as long as there are enough free acid groups left to form a salt with chlorhexidine. Optional neutralization of the polymer having pendant or terminal carboxyl groups converts the carboxyl groups to carboxylate anions, thus having a water-dispersibility enhancing effect. Suitable neutralizing agents include tertiary amines, metal hydroxides, ammonium hydroxide, phosphines, and other agents well known to those skilled in the art. Tertiary amines and ammonium hydroxide are suitable, such as triethyl amine, dimethyl ethanolamine, N-methyl morpholine, and the like, and mixtures thereof. It is recognized that primary or secondary amines may be used in place of tertiary amines, if they are sufficiently hindered to avoid interfering with the chain extension process.

### Other Additives

Other additives, well known to those skilled in the art, may be used to aid in preparation and/or formulation of the dispersions and articles of this disclosure. Such additives include surfactants, defoamers, antioxidants, plasticizers, fillers, rheology modifiers, UV absorbers, light stabilizers, crosslinkers, additional antimicrobial additives (such as antiseptics, bactericides, bacteriocins, disinfectants, and/or preservatives), and the like. In certain embodiments, one or more of the following auxiliary additives can also be added to the compositions described herein: preservatives (such as antimicrobials, algaecides, bactericides, and/or fungicides other than those described herein), stabilizers (such as antioxidants, UV absorbers, and/or anti-hydrolysis agents), solvents, coalescents, plasticizers, humectants, scratch-resistance agents, scrub-resistance agents, mar-resistance agents, antistatic agents, fragrances, aromatic chemicals, colorants, crosslinking agents, anti-foaming agents, flow agents, levelling agents, fluorescent agents, whitening agents, optical brighteners, hydrophobing agents, water-repellent agents, surface modifiers (such as waxes, anti-blocking agents, and/or release agents), slip control agents, pH buffers, coupling agents, adhesion promoters, and wetting agents.

Other antimicrobial additives, some of which may have synergistic effect, include cationic surfactants, metal ions (such as silver and copper), bleach, botulin, triterpenoid-based compounds (such as lanolin), hydrogen peroxide, organic peroxides, peracetic and/or performic acid, iodine and/or iodized compounds, alcohols, phenolic compounds (such as halogenated, quaternary ammonium, phosphonium and/or sulfonium salts), isothiazolinones, permanganate ions, pyridinium bromide polymers, chitosan, tributyltin, eugenol, thymol, carvacrol, triclosan, triclocarban, zinc pyrithione (bacteriostatic), sterols, sterol esters (e.g., lanolin and botulin, oleanolic acid, ursolic acid, squalene and/or triterpenoids derivatives, aldehydes; acids, bases (Ca(OH)₂) and amphoterics which create pH environment hostile to microbes. In certain embodiments, one or more of the following may be included in the compositions described herein: Quaternary ammonium compounds such as dequalinium chloride, benzalkonium chloride, cetyl trimethyl ammonium bromide, di decyldimethylammonium chloride, amine oxide surfactants, benzododecinium bromide, 1-[12-(Methacryloyloxy)dodecyl]pyridinium bromide polymers. Metals and their compounds such as silver and its salts, copper and its salts, zinc oxide, zinc pyrithione, gold, titanium dioxide, tin compounds. Acids and their derivatives such as sorbic acid and sorbates, lactic acid, citric acid, malic acid, benzoic acid and benzoates, tartaric acid and tartrates, geranic acid, acetic acid, cinnamic acid, caffeic acid, 5-aminobarbituric acid, octanoic acid, propionic acid, 3-iodopropanoic acid, salicylic acid, boric acid, 5-aminobarbituric acid. Phenolics and alcohol containing compounds such as isopropanol, ethanol, thymol, eugenol, carvacrol, triclosan, catechins, chlorocresol, carbolic acid, o-phenyl phenol, methylparaben, ethylparaben, propylparaben, butylparaben, benzyl alcohol, glycerin, chlorobutanol, phenyl ethyl alcohol, glycols, triethylene glycol, bromonitropronalediol. Peroxides such as hydrogen peroxides, organic peroxides, performic acid, peracetic acid, persulfates, perborates, perphosphates. Polycyclic compounds based on terpene and sterol such as botulin, lanolin, ursolic acid. Biguanides such as chlorhexidine salts, polyaminopropyl biguanide, polyhexanide, alexidine salts, octenidine salts. Halogen-containing compounds such as N-halamines, fluorine-, chlorine-, and iodine-containing compounds such as povidone, iodides, diiodomethyl p-tolyl sulfone, halogenated phenolic compounds. Aldehydes such as glutaraldehyde, cinnamyl aldehyde, paraformaldehyde. Alkali hydroxides such as calcium hydroxide, manganese hydroxide, sodium hydroxide, potassium hydroxide. Other antimicrobial compounds including tea tree oil, eucalyptus oil, spearmint oil, nisin, benzyl benzoate, isothiazolinones, antraquinone, sodium metabisulfite, sulfur dioxide, levofloxacin, trilocarban, potassium permanganate.

In certain embodiments, the dispersions according to the present subject matter may have total solids of at least about 20 wt. %, such as at least about 25 wt. %, or at least about 30 wt. %.

In certain embodiments, a coating or an article can be prefabricated from an acid-bearing polyurethane, and soaked in and impregnated with the solution of chlorhexidine or other biguanide carbonate. Upon drying, carbonic acid, from which the carbonate counterion originated, decomposes into volatile carbon dioxide and water thus liberating free base of chlorhexidine to form a salt with the polymer.

### EXAMPLES

The following examples provide illustrations of the present subject matter. These examples are non-exhaustive and are not intended to limit the scope of the subject matter.

Test Methods

AATCC TM147

AATCC (American Association of Textile Chemists and Colorists) TM147—Antibacterial Activity: Parallel Streak Method is a qualitative screening test to determine bacteriostatic (antimicrobial) activity of diffusible antimicrobials on treated textiles surfaces. The scope of the test method is to determine bacteriostatic (inhibition of multiplication and growth) activity by diffusion of the antimicrobial agent through agar. The test sample (textile) is placed in intimate contact with a nutrient agar surface which has been previously streaked (parallel streaks) with an inoculum of test organism. After 24 hours incubation, the bacteriostatic activity is demonstrated by a clear area of interrupted growth underneath and along the sides of the test material. AATCC TM 147 is incorporated herein as if fully written out below.

The bacteria used in the AATCC TM147 testing described herein were Klebsiella pneumoniae and Staphylococcus aureus. Klebsiella pneumoniae is a gram negative bacteria belonging to a family which accounts for about 8% of all hospital-acquired infections, such as respiratory and urinary tract infections; it is usually only problematic to those who are immunocompromised, and some members of the family are resistant to antibiotics. Staphylococcus aureus is a gram positive bacteria which is carried by 30% of people, in whom it does not cause problems, but strains may cause blood infections, pneumonia, endocarditis, or osteomyelitis; those with weakened immune systems are at higher risk of infection, and some strains (e.g., MRSA, VISA, VRSA) are resistant to antibiotics.

### Preparing Samples for AATCC TM147

The dispersions described below which were tested according to AATCC TM147 were adjusted to 27.5% solids content. Untreated cotton fabric textile was obtained and cut into strips 5 cm wide and 12 cm long. About 30 g of polymer dispersion to be tested was poured into a petri dish, and, using forceps, one strip at a time was dipped into the polymer dispersion. The textile was submerged in the polymer dispersion and slowly raised by one end to reduce bubble formation. Both sides were thoroughly coated by flipping the sample at least four times. Once sufficiently saturated, the excess polymer dispersion was allowed to drip off and then the textile was placed onto a piece of mylar. The mylar was cut to fit each textile and binder clips were placed on the ends to hold it in place. Some tension was put on the textile by pulling with the forceps and clamping with the binder clips. This was done to prevent the textile from curling, and having bubbles form between the textile and the mylar. (These imperfections could reduce the contact of the polymer and the bacteria, potentially giving skewed results.) The textiles were allowed to cure in a 300° F. oven for 3 minutes. The binder clips were removed, the samples were cut into 2.5 cm×5 cm rectangles, and mylar removed.

Leaching Procedure

For the samples described below which were subjected to the leaching procedure, samples as described above with regard to preparing samples for AATCC TM147 were leached in demineralized (“DM”) water before testing to determine if the antimicrobial agent was adequately adhered to the polymer, and to ensure that the antimicrobial effects were the result of the polymer and not due to leaching. Samples that were leached in DM water were prepared as follows: Samples were removed from their mylar and placed into 2 gallon buckets of DM water (one sample per bucket). The bucket was under gentle agitation using a mixer and the water was changed every 3 hours. At each water change the samples were taken out and placed on mylar, the buckets were rinsed and wiped out, refilled, and the samples were reintroduced. To reduce the chances of contamination, forceps were rinsed and scrubbed after touching samples that contained different polymers/antimicrobial agents. Leaching times varied but the average was 170 hours. The textiles were allowed to air dry before placing in plastic bags. After completely drying, the samples were cut into 2.5 cm×5 cm rectangles.

Certain samples, as described below, were soaked in sodium lauryl sulfate (“SLS”) solution. Certain samples, as described below, were leached in DM water using the procedure above before being soaked in the SLS, while other samples, as described below, were only soaked in SLS. 900 g of 0.5 SLS solution were used for each sample; fresh SLS solution was used for each sample.

JIS-Z-2801

The Japanese Industrial Standard (JIS)-Z-2801 test method is designed to evaluate the antibacterial activity of a variety of surfaces including plastics, metals and ceramics. Two types of bacteria are used to challenge the test surfaces: Staphylococcus aureus and Escherichia coli. Each test specimen (50 mm×50 mm) is placed in a petri dish and the test inoculum is added onto the specimen. A film is then added to cover the entire test specimen. Triplicate specimens are inoculated for each data point. Immediately after inoculation, untreated specimens are processed to count viable organisms at Time 0. Untreated and treated specimens are then incubated at 35° C. for 24 hours. Test organisms are enumerated by washing specimens in a neutralizing broth and plating using serial dilutions. JIS-Z-2801 is incorporated herein by reference as if fully written out below.

### Preparing Samples for JIS-Z-2801

Mylar film was cut into 5 cm×5 cm squares and rinsed well under DM water, dried with paper towels, and allowed to air dry before being coated. The desired polymer was pipeted onto the mylar squares and drawn down using a 6 mil wet film applicator rod. The squares were immediately moved to another piece of mylar to prevent the back from getting wet with polymer. After the coated mylar samples were air dried, they were put into a 300° F. oven for three minutes. A sticker was placed on the uncoated side to make sure that the correct side would be tested. Coated mylar samples were placed into plastic jars instead of plastic bags because the coated surface was sticking to the bag and the other samples. When placed in the jars they only touch the uncoated sides and can stand vertically to prevent disruption of the coated surface.

### Preparation of Polymer 1

Polymer 1 was prepared according to the following procedure: 120 grams of polyether-1,3-diol (Ymer N120 from Perstop), 120 grams of Polytetrohydrofuran polyether glycol Mₙ˜1000 g/mol (Tarathane 1000 from The Lycra Company), 17.5 grams of Dimethylolpropanoic Acid (DMPA® from GEO Specialty Chemicals), 210 grams of methylene-bis-(4-cyclohexylisocyanate) (Desmodur W from Covestro) were charged into a vessel equipped with a mechanical stirrer and thermocouple under Nitrogen gas and heated to 225° F. Reaction was monitored by determining the amount of free isocyanate using dibutylamine (Acros Organics) back titration (ASTM D1638). When the desired amount of free isocyanate is left over, the vessel is cooled to 150° F. and the triethylamine (Millipore Sigma) is charged. The resulting prepolymer was stirred thoroughly and dispersed into water. It was then promptly chain-extended with diluted hydrazine, and the amount of NCO is tracked through IR spectroscopy until there is no more free isocyanate left in solution. Total solids of the polymer dispersions, if reported, were obtained by evaporation of water using a ventilated oven.

### Preparation of Polymer 2

Polymer 2 was prepared the same as Polymer 1, without the addition of Dimethylolpropanoic Acid.

### Preparation of Polymer 3

Polymer 3 was Carboset® CR-765 polymer available from Lubrizol Advanced Materials, Inc.

### Preparation of Polymer 4

Polymer 4 was Sancure® 825 polymer available from Lubrizol Advanced Materials, Inc.

### Preparation of Salts

The Polymer dispersion mentioned below with regard to each specific Salt was adjusted to a total solids amount of 27.5%, and chlorhexidine (or other ingredient, as specified) was added in the percentage by weight identified below with regard to each specific Salt, based on the dry weight of the polymer. The Salts described below were prepared by adding the appropriate amount of chlorhexidine a vessel first, followed by adding the polymer dispersion. The vessel was stirred for four hours, followed by filtration to ensure all the chlorhexidine went into solution.

Salt 1 was made using Polymer 1 with 1 wt. % chlorhexidine free base.

Salt 2 was made using Polymer 1 with 0.1 wt. % chlorhexidine free base.

Salt 3 was made using Polymer 1 with 2.5 wt. % chlorhexidine free base.

Salt 4 was made using Polymer 1 with 6 wt. % chlorhexidine free base.

Salt 5 was made using Polymer 1 with 10 wt. % chlorhexidine free base.

Salt 6 was made using Polymer 1 with 5 wt. % chlorhexidine free base.

Salt 7 was made using Polymer 1 with 10.4 wt. % chlorhexidine free base.

Salt 8 was made using Polymer 2 with 10 wt. % chlorhexidine free base.

Salt 9 was made using Polymer 2 with 10 wt. % chlorhexidine dihydrochloride.

Salt 10 was made using Polymer 2 with 10 wt. % 1,3-diphenylguanidine.

Salt 11 was made using Polymer 2 with 10 wt. % aminoguanidine bicarbonate.

Salt 12 was made using Polymer 2 with 10 wt. % guanidine hydrochloride.

Salt 13 was made using Polymer 2 with 10 wt. % Reputex® (polyhexamethylene biguanide) from Vantocril.

Salt 14 was made using Polymer 2 with 1 wt. % chlorhexidine free base.

Salt 15 was made using a blend of 80% Polymer 3 and 20% Polymer 1 by weight, with 1 wt. % chlorhexidine free base, based on the total weight of Polymers 1 and 3.

Salt 16 was made using a blend of 80% Polymer 4 and 20% Polymer 1 by weight, with 1 wt. % chlorhexidine free base, based on the total weight of Polymers 1 and 4.

Control 1 was Caliwel™ Industrial Antimicrobial Coating for Behind Walls and Basements.

Control 2 was Sherwin-Williams Paint Shield® Microbial Interior Latex Paing.

Table 1 reports results of samples tested according to AATCC TM147, prepared as described above, as follows. Example 1 included salt 1, Example 2 included Salt 2, Example 3 included Polymer 1 (unsalted), Example 4 included Salt 3, Example 5 included Salt 4, and Example 6 included Salt 5.

Table 1 indicates whether there was growth (yes or no) on each Example and the zone of inhibition (“Zone”, in mm), as tested using Klebsiella pneumoniae (“K.p.”) and Staphylococcus aureus (“S.a.”), according to AATCC TM147.

TABLE 1

| | |
|---|---|
| K.p. | S.a. |

| | Growth | Zone | Growth | Zone |
|---|---|---|---|---|
| Example 1 | No | 0 | No | 0 |
| Example 2 | Yes | 0 | Yes | 0 |
| Example 3 | Yes | 0 | Yes | 0 |
| Example 4 | No | 4 | No | 5 |
| Example 5 | No | 7 | No | 7 |
| Example 6 | No | 6 | No | 8 |

Table 2 reports results of samples tested according to AATCC TM147, prepared as described above, as follows. Example 7 included Control 1, Example 8 included Control 2, Example 9 included Polymer 1 (unsalted), Example 10 included Salt 6, Example 11 included Salt 7, Example 12 included Salt 8, Example 13 included Salt 9, Example 14 included Salt 10, Example 15 included Salt 11, Example 16 included Salt 12, and Example 17 included Salt 13.

Table 2 indicates whether there was growth (yes or no) on each Example and the zone of inhibition (“Zone”, in mm), as tested using Klebsiella pneumoniae (“K.p.”) and Staphylococcus aureus (“S.a.”), according to AATCC TM147.

TABLE 2

| | |
|---|---|
| K.p. | S.a. |

| | Growth | Zone | Growth | Zone |
|---|---|---|---|---|
| Example 7 | Yes | 1 | Yes | 2 |
| Example 8 | No | 5.5 | No | 5.75 |
| Example 9 | Yes | 0 | Yes | 0 |
| Example 10 | No | 2.5 | No | 2.5 |
| Example 11 | No | 3.75 | No | 2.75 |
| Example 12 | No | 2 | No | 4.5 |
| Example 13 | No | 1 | Yes | 2.25 |
| Example 14 | No | 0.75 | No | 0 |
| Example 15 | Yes | 0 | No | 1 |
| Example 16 | Yes | 0 | No | 1 |
| Example 17 | No | 2.5 | No | 3.5 |

With regard to Example 7, although growth occurred on the surface of the textile, a zone of inhibition was still created in the surrounding media.

Table 3 reports results of samples tested according to AATCC TM147, prepared as described above, as follows. Example 18 included Salt 1, and was not leached. Example 19 included Salt 1, and was leached in DM water as described above. Example 20 included Salt 14 and was not leached. Example 21 included Salt 14, and was leached in DM water as described above.

Table 3 indicates whether there was growth (yes or no) on each Example and the zone of inhibition (“Zone”, in mm), as tested using Klebsiella pneumoniae (“K.p.”) and Staphylococcus aureus (“S.a.”), according to AATCC TM147.

TABLE 3

| | |
|---|---|
| K.p. | S.a. |

| | Growth | Zone | Growth | Zone |
|---|---|---|---|---|
| Example 18 | No | 0 | No | 0 |
| Example 19 | No | 0 | No | 0 |
| Example 20 | No | 1 | No | 2.1 |
| Example 21 | No | 0 | No | 0.75 |

Table 4 reports results of samples tested according to AATCC TM147, prepared as described above, as follows. Example 22 included Salt 15, and was not leached. Example 23 included Salt 16, and was not leached. Example 24 included Salt 15 and was leached in DM water as described above. Example 25 included Salt 16 and was leached in DM water as described above.

Table 4 indicates whether there was growth (yes or no) on each Example and the zone of inhibition (“Zone”, in mm), as tested using Klebsiella pneumoniae (“K.p.”) and Staphylococcus aureus (“S.a.”), according to AATCC TM147.

TABLE 4

| | |
|---|---|
| K.p. | S.a. |

| | Growth | Zone | Growth | Zone |
|---|---|---|---|---|
| Example 22 | No | 0 | No | 0.5 |
| Example 23 | No | 0 | No | 0 |
| Example 24 | No | 0 | No | 0 |
| Example 25 | No | 0 | No | 0 |

Table 5 reports results of samples tested according to AATCC TM147, prepared as described above, as follows. Example 26 was tested using sanded stainless steel as the test sample. Example 27 included Salt 1 and was soaked in SLS solution and leached in DM water, as described above.

Table 5 indicates whether there was growth (yes or no) on each Example and the zone of inhibition (“Zone”, in mm), as tested using Klebsiella pneumoniae (“K.p.”) and Staphylococcus aureus (“S.a.”), according to AATCC TM147.

TABLE 5

| | |
|---|---|
| K.p. | S.a. |

| | Growth | Zone | Growth | Zone |
|---|---|---|---|---|
| Example 26 | Yes | 0 | Yes | 0 |
| Example 27 | No | 0 | No | 0 |

Example 28 and 29 were tested according to JIS-Z-2801, in which the samples were measured for bacterial load compared to an internal control. Example 28 included Salt 1 and Example 29 was uncoated mylar film as a negative control. Example 28 showed 99.98% reduction (3.76 logarithmic reduction) in cells/cm² after 24 hours, as compared with the internal control. Example 29 showed 82.89% reduction (0.77 logarithmic reduction) in cells/cm2 after 24 hours, as compared with the internal control.

Examples 30 through 33 were tested according to JIS-Z-2801, in which the samples were measured for bacterial load compared to an internal control. Example 30 included Salt 1. Example 31 included Salt 1, and was soaked in SLS solution as described above. Example 32 included Salt 1. Example 33 included Salt 1, and was soaked in SLS solution as described above.

Example 30 showed 17.8% reduction (0.09 logarithmic reduction) in cells/cm² after 10 minutes, as compared with the internal control. Example 31 showed 21.6% reduction (0.11 logarithmic reduction) in cells/cm² after 10 minutes, as compared with the internal control. Example 32 showed 61.5% reduction (0.41 logarithmic reduction) in cells/cm² after 6 hours, as compared with the internal control. Example 33 showed no reduction after 6 hours, as compared with the internal control. A comparison between Examples 33 and 34 shows that the antimicrobial mechanism for the polyurethanes salted with chlorhexidine comes from the chlorhexidine.

### Example of Anionic Polyurethane Dispersion Salted with Chlorhexidine

The following materials are charged to a reactor equipped with a mechanical stirrer, thermocouple, and dry nitrogen flow: 305 grams polypropylene glycol with Mₙ˜1,000 g/mol, 35 grams dimethylolpropanoic acid, 245 grams isophorone diisocyanate, and 0.02 grams stannous octoate (FASCAT™ 2003 from Elf Atochem North America). The stirrer is then turned on, and the mixture is heated to 90° C. and stirred at this temperature for ˜2 hours. The resulting prepolymer is cooled to about 70° C., and 16 grams of triethylamine are gradually added. After ˜10 minutes of mixing, 400 grams of the prepolymer are charged with good mixing over 5 minutes to a vessel containing 700 grams DI water at 15° C. The resulting dispersion is stirred for ˜15 minutes and then chain-extended by adding, over 10 minutes, 22 grams of 35% solution of hydrazine. The dispersion is then covered and mixed overnight, followed by adding 26 grams chlorhexidine free base, and stirring the mixture overnight at ambient temperature. The resulting product is an anionic polyurethane dispersion salted with chlorhexidine having molar ratios COOH:TEA:CHX of 1:0.6:0.2.

It is contemplated that the compositions described herein may be useful in the following areas of application:

Consumer and Personal: Clothing, footwear, cosmetics, soap and lotion dispensers, shower caddy, spatula, can opener, cell phones, remote controls, towels, napkins, toothbrushes, deodorant, shower tiles, sinks, microwave and oven buttons, computers, electronic consoles and devices, luffas, towels, other high touch surfaces.

Household: Paints, coatings, varnishes, appliances, doorknobs, handrails, flooring, towels, upholstery, seating, rugs, carpets, doormats, handrails, other high touch surfaces.

Institutional and Commercial: Control panels, gyms, offices, shared seating and waiting areas, communal equipment, portable restrooms, filtration media water purification, community pools, locker rooms, lockers, community and public sectors parks and picnic areas.

Food: Utensils, countertops, conveyor belts, packaging, flooring, kitchen accessories, tablecloths and reusable napkins, commercial food & drink preparation.

Medical: Masks, gloves, face shields, beds, general personal protective equipment, bedding, curtains, surgical equipment, medical devices, instrumentation, flooring, hard surfaces, waiting room furniture, check in kiosks, computers.

Hospitality: Bedding, toiletries, doorknobs and handles, desks, kitchen equipment, televisions and remotes, elevators (buttons), cruise ships, towels, tanning chairs.

Transportation: Seating (upholstery), railings, hard surfaces, handles, seat belts, security boxes during flight check in, shareable transportation (scooters, bikes, motorized bikes).

Education: Desks, cafeteria seats and tables, lockers, day cares, toys, jungle gyms, recess equipment.

Entertainment: Common area seating (arenas, stadiums, theaters, etc.) amusement ride seating, ATMs, casino tables, casino chips, tanning beds.

Except in the Examples, or where otherwise explicitly indicated or required by context, all numerical quantities in this description specifying amounts of materials, reaction conditions, molecular weights, number of carbon atoms, and the like, are to be understood as modified by the word “about”. It is to be understood that the upper and lower amount, range, and ratio limits set forth herein may be independently combined, and that any amount within a disclosed range is contemplated to provide a minimum or maximum of a narrower range in alternative embodiments (with the proviso, of course, that the minimum amount of a range must be lower than the maximum amount of the same range). Similarly, the ranges and amounts for each element of the subject matter disclosed herein may be used together with ranges or amounts for any of the other elements.

While certain representative embodiments and details have been shown for the purpose of illustrating the subject matter disclosed herein, it will be apparent to those skilled in this art that various changes and modifications may be made therein without departing from the scope of the subject matter. In this regard, the scope of the invention is to be limited only by the following claims.

1. A polyurethane composition comprising a polyurethane with at least one free acid group salted with a biguanide free base.

2. The composition of claim 1, wherein the at least one free acid group comprises at least one of carboxylic acid, sulfonic acid, or phosphonic acid.

3. The composition of claim 1, wherein the biguanide free base comprises a bisbiguanide free base.

4. The composition of claim 1, wherein the biguanide free base comprises at least one of chlorhexidine free base, alexidine free base, polyhexanide free base, or polyaminopropyl biguanide free base.

5. The composition of claim 1, wherein the polyurethane comprises the reaction product of:
a. a polyisocyanate component having on average two or more isocyanate groups;
b. a poly(alkylene oxide) tethered and/or terminal macromonomer, wherein the alkylene of the alkylene oxide has from 2 to 10 carbon atoms, wherein the macromonomer has a number average molecular weight of at least 300 g/mole and one or more functional reactive groups characterized as active hydrogen groups, the reactive groups primarily at one end of the macromonomer, such that the macromonomer has at least one non-reactive end, and at least 50 wt. % of the alkylene oxide repeat units of the macromonomer are between the non-reactive end of the macromonomer and the closest reactive group of the macromonomer to the non-reactive terminus;
c. an isocyanate-reactive compound having at least one free acid group; and
d. optionally at least one active-hydrogen containing compound other than (b) or (c).

6. The composition of claim 1, wherein the polyurethane has from 12 wt. % to about 80 wt. % of alkylene oxide units present in the poly(alkylene oxide) macromonomer.

7. The composition of claim 1, wherein the at least one free acid group is salted with a biguanide free base to create an ionic salt bond between the at least one free acid group and the biguanide.

8. The composition of claim 1, wherein the molar ratio of biguanide to the at least one free acid group is from 1.2:1 to 0.1:1.

9. The composition of claim 1, wherein the at least one free acid group is present in the polyurethane at a concentration of from 0.002 to 5 millimoles/gram of polyurethane before being salted with the biguanide free base.

10. The composition of claim 1, wherein the biguanide free base is present in the composition at an amount of from 0.25 to 10 wt. %, based on the total weight of the polyurethane.

11. The composition of claim 1, wherein the polyurethane has from 40 to 80 wt. % alkylene oxide repeat units present in repeat units of the macromonomer.

12. The composition of claim 1, wherein the poly(alkylene oxide) chains of the macromonomer have number average molecular weights from about 88 to 10,000 g/mole.

13. The composition of claim 1, wherein the poly(alkylene oxide) chains of the macromonomer have at least 50% ethylene oxide units based on their total alkylene oxide units.

14. A coating comprising the composition of claim 1, used as a coating on a surface.
